(19) United States
(12) Patent Application Publication
(10) Pub. No.: US 2006/0247543 A1
(43) Pub. Date: Nov. 2, 2006

(54) High resoution bio-impedance device

(76) Inventors: Bruce Cornish, Brisbane (AU); Brian Thomas, Queensland (AU); Scott Chetham, Teneriffe (AU)

Correspondence Address: ROBERT A. PARSONS, 4000 N. CENTRAL AVENUE, SUITE 1220, PHOENIX, AZ, 85012, US

(21) Appl. No.: 10/530,860

(22) PCT Filed: Oct. 9, 2003
(86) PCT No.: PCT/AU03/01333

§ 371 Date: May 25, 2006

(30) Foreign Application Priority Data
Oct. 9, 2002 (AU) 2002951925

(51) Int. Cl. A61B 5/02

(52) U.S. Cl. 600/508; 600/547

(57) ABSTRACT

A method and apparatus for the non-invasive measurement of cardiac function. A signal is applied between a pair of electrodes on a patient. The signal delivers a constant alternating current at multiple simultaneous frequencies. A second pair of electrodes measures a voltage signal. The impedance at each frequency is obtained by demodulating the current signal and the voltage signal using techniques such as Fast Fourier Transform (FFT). The FFT gives a phase and amplitude which is converted to an impedance value. The impedance values are fitted to a theoretical frequency dependent impedance locus and the locus is extrapolated to obtain a value at zero frequency. The steps are repeated to obtain a time-varying plot of impedance and measures of cardiac function are calculated from the time-varying plot.

### TECHNICAL FIELD

The present invention relates to a device for measuring a biological parameter such as extracellular fluid in a person and in particular to a non-invasive bio-impedance device for accurately measuring the cardiac output of a person using impedance measurements at multiple frequencies of stimulation.

### BACKGROUND OF THE INVENTION

Cardiovascular disease is the greatest health problem in the developed world, accounting for greater than 40% of all deaths. The economic effects of heart disease and stroke, the principle components of cardiovascular disease, on health care systems grow larger as the population ages. Billions of dollars are spent on the treatment and rehabilitation of cardiac patients.

The electrocardiogram (ECG) measures electrical activity of the heart and therefore provides useful information concerning the sequence and pattern of muscular activity of the heart chambers. The ECG does not evaluate, however, the efficiency of the heart as a pump, i.e., it does not show the amount of blood being pumped through the cardiovascular system.

The cardiac output (CO), a quantitative measure of blood flow, is one of the most useful parameters in assessing cardiac capability and is the volume of blood pumped by each ventricle per minute. CO is determined by multiplying the heart rate (HR) and stroke volume (the volume of blood ejected during each ventricular contraction) and is measured in L/minute.

The assessment of CO is an essential aspect of haemodynamic monitoring which is necessary in numerous clinical situations including the rehabilitation of patients over extended periods following discharge from hospital. CO is also one indicator used in the assessment of cardiovascular fitness of healthy individuals in training (for example, athletes, military personnel and fire-fighters). However, it is one of the most difficult parameters to measure.

The most accurate and reliable methods of measuring CO are extremely invasive and require direct access to the arterial circulation using catheters. These techniques expose the patient to pain, risk of infection, disease transmission, risk of bleeding or thrombosis, and the techniques are expensive, time consuming and normally can not be performed outside a hospital. The only reliable non-invasive method is that of echocardiography using ultrasound. However, this procedure requires major facilities, expert operator skills and incurs very high costs.

Impedance cardiography is a non-invasive method which has the potential for monitoring the mechanical activity of the heart with minimised risk to the patient. However, the relatively poor sensitivity and the inaccuracy of the current methods of impedance cardiography severely limit its application.

The acquisition of a portable, accurate and reliable impedance cardiograph, at an affordable price, would enable GPs to perform complete cardiac assessments on their patients and obtain immediate and vital physiological data. At present this information can only be obtained by referring the patient to a hospital or a major medical facility with an expert cardiac sonographer, which make take days or weeks.

U.S. Pat. No. 5,309,917, in the names of Wang and Sun, describes a system and method of continuous cardiac monitoring in which thoracic impedance and ECG signals are gathered and processed. Current injection and recording pairs of electrodes are applied to a patient's skin and a variable alternating current is applied to the patient through the injecting electrodes. The recording electrodes are provided to sense voltage levels on the patient from which thoracic impedance is determined.

A pre-processor excites the current injecting electrodes at high frequency (100 kHz) and low amplitude (up to 4 mA RMS) alternating current. The pre-processor outputs four analogue signals: the mean thoracic impedance signal (Z0), the change in thoracic impedance signal (delta Z or ΔZ), the time-derivative impedance signal (dZ/dt) and the electrocardiogram signal (ECG). The time-derivative impedance signal is converted to the frequency domain to determine cardiac events, stroke volume and cardiac output.

A major drawback of the above method and system is a single frequency is used to measure impedance at the electrodes. The use of a single high frequency (eg 50 kHz to 100 kHz) presents inaccuracies in determining cardiac activity and output, as current at this frequency passes through both intra- and extra-cellular fluids. Blood plasma is purely extracellular fluid.

Another system is described in U.S. Pat. No. 6,339,722 in the name of Heethaar et al. Unlike the system above, the patent describes an apparatus for measuring a biological parameter, such as cardiac output, using a current source generating two signals of different frequencies. The current source is provided with a galvanic separation in relation to the recording part of the instrument to reduce interference effects caused by electromagnetic radiation at high frequencies of stimulation. A stimulating current with constant amplitude is provided at a low frequency and a high frequency of stimulation, in a frequency range of up to 2000 kHz. Changes in voltage within the stimulated body region are recorded by a recording pair of electrodes, and the measured voltage is transformed into a bio-impedance signal. The use of two frequencies of stimulation provides independent measurements since the low frequency currents are transmitted mainly through the extracellular fluid and the high frequency currents are transmitted through both extracellular and intracellular fluid. While the low frequency current of this device passes mainly through the extracellular fluid it still penetrates the intracellular component and hence has limited sensitivity. Also being a single measurement it has inherent limited accuracy and precision.

A common drawback of the above systems is the use of current sources to generate the alternating current (AC) at the current injecting electrodes. Current signals generated by current source generators at high frequencies of AC normally have large artefacts that mask the bio-impedance signals. This prevents measurement of the bio-impedance signals.

Another drawback of the above systems is that the bio-impedance signals recorded are a combined measure of intracellular and extracellular fluids, rather than only blood volumes, thereby diminishing the accuracy of the measurement of ventricular ejection of blood (cardiac output). A further limitation is the limited accuracy inherent in results derived from single data points (at single frequencies).

### OBJECT OF THE INVENTION

It is an object of the invention to provide an instrument that measures cardiac output accurately and reliably using non-invasive techniques and does not require expert operator skills.

It is a further object of the invention to provide a portable bio-impedance device for measuring extracellular fluids (blood volume) only in a person or an animal.

### SUMMARY OF THE INVENTION

In one form, although it need not be the only or indeed the broadest form, the invention resides in a method of determining measures of cardiac function in a patient comprising the steps of:
(I) generating an alternating current signal at multiple simultaneous frequencies from a constant current source electrically isolated from the patient;
(II) applying the current to an outer pair of electrodes on the patient;
(III) measuring a voltage signal across an inner pair of electrodes on the patient;
(IV) demodulating the current signal and voltage signal to extract signals at each of said multiple frequencies
(V) determining impedance at each said frequency at a time;
(VI) fitting said impedance at each frequency to a theoretical frequency dependent impedance locus;
(VII) extrapolating the locus to obtain a value of impedance at zero frequency at said time;
(VIII) repeating steps (v) to (vii) to obtain a time varying plot of impedance; and
(IX) calculating volume of extracellular fluid in the patient from said time varying plot.

In a preferred form the steps of demodulating and determining an impedance at a time, comprises the steps of:

sampling the impedance signals to obtain a sampled impedance;

applying a time to frequency domain transform to said sampled signal to obtain transformed impedance signals; and

filtering the transformed impedance signals and isolating each frequency to determine the impedance for each frequency at each time.

Preferably, the change in the impedance value over time and the rate of change in the measured impedance signal dZ/dt is used to determine impedance parameters to calculate cardiac output of said patient In another form of the invention there is provided an apparatus for non-invasive measurement of cardiac function in a patient, said apparatus comprising:

a constant current source, electrically isolated from said patient, generating an alternating current signal at multiple simultaneous frequencies, which is applied to an outer pair of electrodes on a patient;

an inner pair of electrodes applied to a patient for measuring a voltage signal;

signal processing means for converting said applied current signal and measured voltage signal to impedance signals at each frequency at a time;

means for determining impedance values at a zero frequency (Z₀) and at infinite frequency (Z_{inf}) at a plurality of time intervals; and

means for calculating measures of cardiac function in said patient from said impedance values.

Preferably a time derivative of said impedance signal is mathematically obtained using the extrapolated impedance at zero frequency (Z₀) or at infinite frequency (Z_{inf}).

### BRIEF DESCRIPTION OF THE DRAWINGS

FIG. 1 is a circuit diagram of an electric circuit modelling a biological tissue.

FIG. 2 is a flow chart showing the process steps for obtaining bio-impedance signals and measuring extracellular fluid in accordance with an embodiment of the invention.

FIG. 3 is a Cole-Cole plot of impedance signal data over a range of frequencies.

FIG. 4 is a trace showing measured impedance over time, the time derivative dZ/dt of impedance trace and the corresponding ECG trace.

FIG. 5 is a schematic diagram showing an apparatus for obtaining bio-impedance signals and measuring extracellular fluid in accordance with an embodiment of the invention.

FIG. 6 is a block diagram showing elements of a signal generator.

FIG. 7 is a block diagram showing elements of a signal receiver.

FIG. 8 is a block diagram showing elements of a signal processing unit.

### DETAILED DESCRIPTION OF THE PREFERRED EMBODIMENT

For the purposes of this invention, by “patient” is meant a person or animal.

In a preferred form, the invention will be described with reference to a bio-impedance device for measuring aspects of cardiac function, such as the stroke volume, cardiac output, cardiac index, heart rate, pre-ejection time, and left ventricular ejection time. However, it should be noted that the invention could also be realised to measure other biological parameters relating to bodily fluids, such as thoracic fluid content, ejection fraction, pulmonary wedge pressure and systolic time ratio.

There are several invasive methods available for assessing heart function, many of which involve the use of venous or arterial catheters into, or in very close proximity to the cardiac chambers (eg thermo- or dye-dilution).

Impedance cardiography is a completely non-invasive technique that can measure cardiac pumping performance on a beat-by-beat basis. The technique can be performed on virtually all subject groups including the critically ill, elderly, very young or pregnant individuals. However, its correlation and agreement with other techniques has been reported as less than ideal and it generally overestimates the cardiac output particularly in clinical subjects (Spiering et al, “Comparison of impedance cardiography and dye dilution method for measuring output”, Heart, 1998; 79(5): 437, 441).

The theory behind bioelectrical impedance can be explained in relation to a conducting cylinder. The impedance of a conducting cylinder is related to the conductor length, cross sectional area, and signal frequency. Using a constant signal frequency the impedance is given by:

$$Z = \frac{ρ L}{A}$$

where Z=impedance (Ω)
ρ=resistivity of the medium (Ω cm)
L=conductor length (cm)
and A=cross sectional area (cm²)
Using V=volume (cm³)=A×L and eliminating A
Yields:

$$V = \frac{ρ {L}^{2}}{Z} \qquad equation 1$$

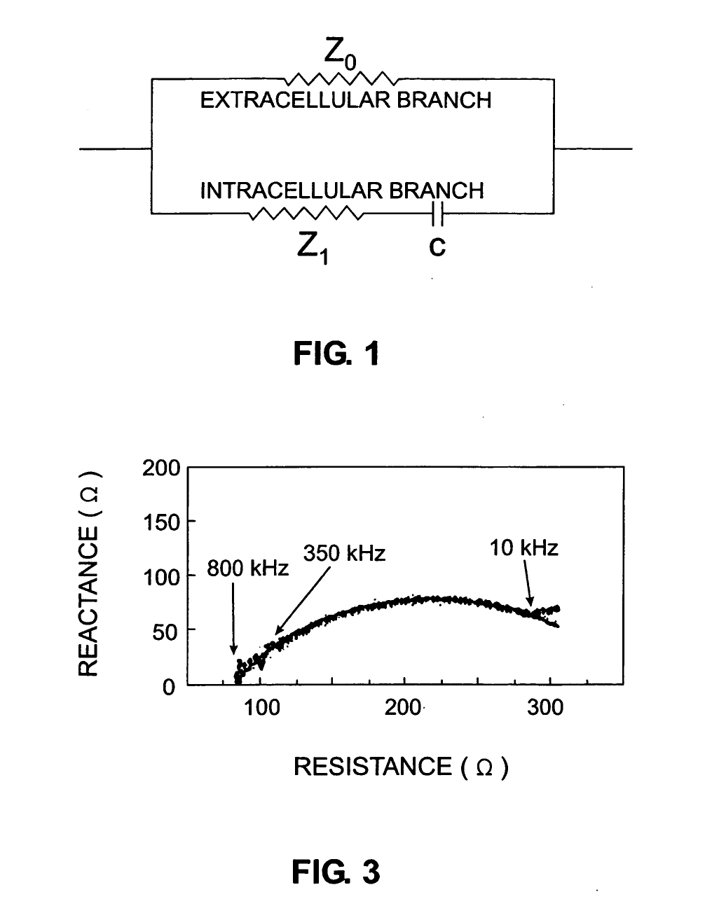

Referring now to FIG. 1, there is shown a simple equivalent circuit representing biological tissue. The extracellular current pathway is purely resistive, while the intracellular current pathway has an associated capacitance due to the cell membrane. The relative magnitudes of the extracellular and intracellular components of an alternating current (AC) are frequency dependent. At zero frequency the capacitor acts as an insulator and all of the current passes through the extracellular fluid. Hence the measured impedance, Z₀, at zero frequency is the impedance of the extracellular fluid. At higher frequencies the capacitor has a finite impedance and the current passes through both branches of the parallel circuit model. The measured impedance at these non-zero frequencies is therefore due to both the extracellular and intracellular fluid volumes.

The volume in equation I is the volume of the conducting medium. If there are changes in the volume of the conducting medium with time, as is the case of continuously varying blood volumes in the region of the heart, then the change in conducting volume is related to the change in impedance by the following equation discussed by Geddes et al in “Principles of applied biomedical instrumentation”: John Wiley & Sons, 1989, New York:

$$Δ V = - 〈 \frac{ρ {L}^{2}}{{Z}_{B}^{2}} 〉 Δ Z \qquad equation 2$$

ΔV=blood volume change
ρ=resistivity of blood
L=distance between measurement electrodes
Z_{B}=baseline impedance value
ΔZ=change in impedance (attributable to stroke volume)

The frequency commonly used in impedance cardiography systems is generally selected between 70 and 100 kHz.

An important parameter of heart function is the stroke volume (SV) (volume of blood ejected during each ventricular contraction) of the heart. Stroke volume can be determined by manipulating equation 1 as was developed by Kubicek et al. in: “Development and evaluation of an impedance cardiac output system”, Aerospace Medicine, 1966; 37:1208, 1212. The stroke volume is represented as:

$$SV = \frac{ρ {L}^{2} {〈 \frac{ⅆ Z}{ⅆ t} 〉}_{max} VET}{{Z}_{B}^{2}} \qquad equation 3$$

where: SV=stroke volume
(dz/dt)ₘₐₓ=maximum rate of change in measured impedance at the beginning of systolic cycle.
VET=left ventricular ejection time.

This technique requires the accurate measurement of the inter-electrode distance placed on a person and also the measurement of haematocrit to determine blood resistivity. A modification to this algorithm was introduced by Bernstein DP: “A new stroke volume equation for thoracic electrical bio impedance: theory and rationale”, Critical Care Medicine, 1986; 14:904,909. This relationship is currently used by the majority of impedance cardiography instruments.

$$SV = \frac{ {L}^{t 3} {〈 \frac{ⅆ Z}{ⅆ t} 〉}_{max} VET}{{Z}_{B}} \qquad equation 4$$

where: L′=thoracic length estimated from the subject's height and weight using a nomogram, L′ also accounts for blood resistivity.

The overall impedance of the thorax varies between subjects. The quoted range is 20 to 48 Ω at frequencies between 50 kHz and 100 kHz. The variation in transthoracic impedance due to the cardiac cycle is approximately 1% of the overall impedance of the thorax (Critchley, L. A. H. in “Impedance cardiography, the impact of a new technology”, 1998, Anaesthesia 53: 677-684). This leads to a very ‘fragile’ signal with a very low signal to noise ratio.

Precise identification of the impedance signal, is essential if accurate measurements of both dZ/dtₘₐₓ and ventricular ejection time are to be made. As noted above, signal to noise ratio in present systems is very low which leads to inaccuracies when these parameters are measured. The problem is exacerbated when the patient moves or exercises. The signal also can be masked by the stimulus artefact and therefore precise positioning of the current injecting and recording electrodes is required to reduce the stimulus artefact to a minimum size.

The most important significant aspect of the accuracy of transthoracic electrical bio-impedance measurements resides in the signal processing of the measured bio-impedance signal.

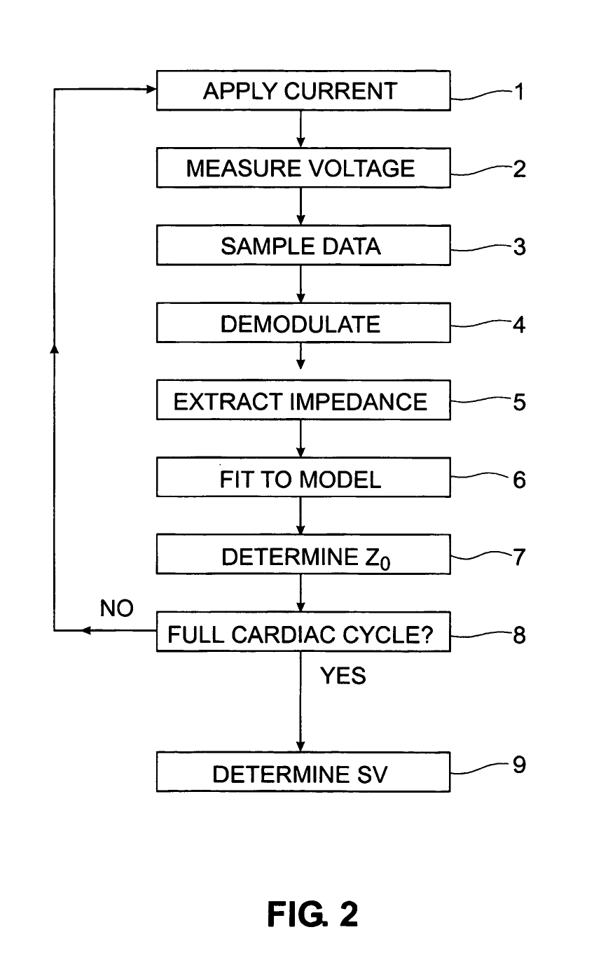

The method of determining stroke volume from bio-impedance data is set out broadly in FIG. 2. A constant current signal at multiple frequencies is applied (step 1) to a pair of outer electrodes positioned on a patient in the thoracic and neck region.

The signal is applied at a number of frequencies simultaneously (at least three but most usefully five or more) in the range 2-2000 kHz. In compliance with Australian standards the applied signal has a maximum voltage of 32 V and a maximum current of 100 μA at 10 kHz. This current limit increases to an upper threshold of 1 mA at 1000 kHz.

A potential difference (voltage) is measured (step 2) between an inner pair of electrodes. The acquired signal will be a superposition of signals at each applied frequency of the current signal. The applied signal and the measured signal are recorded (step 3) and demodulated (step 4) to obtain applied and recorded signals at each frequency.

The distance between the inner pair of electrodes is measured and recorded. The height, weight, age and sex of the patient may also be recorded.

One suitable method of demodulation is to use a fast Fourier transformer (FFT) algorithm to transform time sequence data to the frequency domain. Other digital and analogue demodulation techniques will be known to persons skilled in the field.

Impedance measurements are determined (step 5) from the signals at each frequency by comparing the measured voltage signal to the applied current signal. The FFT algorithm will produce a phase and amplitude for the measured signal compared to the applied signal. The phase and amplitude is used to calculate resistance (R=zsinφ) and reactance (X=zcosφ) at each frequency. A suitable calibration of the amplitude is required to obtain the complex impedance z.

The resistance R(f) and reactance X(f) are frequency dependent according to the Cole-Cole relationship:

$$Z = {R}_{∞} + \frac{Ro - {R}_{∞}}{1 + {( j ω τ )}^{( 1 {-}_{α} )}}$$

It is known that the impedance at zero frequency Z₀ and at infinite frequency Z_{inf} can be determined from a Cole-Cole plot (shown in FIG. 3) by fitting the measured resistance and reactance at each frequency to the theoretical locus (step 6). The locus is then extrapolated to obtain Z₀ and Z_{inf} at the x-axis (step 7).

This process (steps 1-7) is repeated until sufficient impedance data has been compiled to record at least one cardiac cycle (step 8). In practice, multiple cardiac cycles are required for accurate analysis.

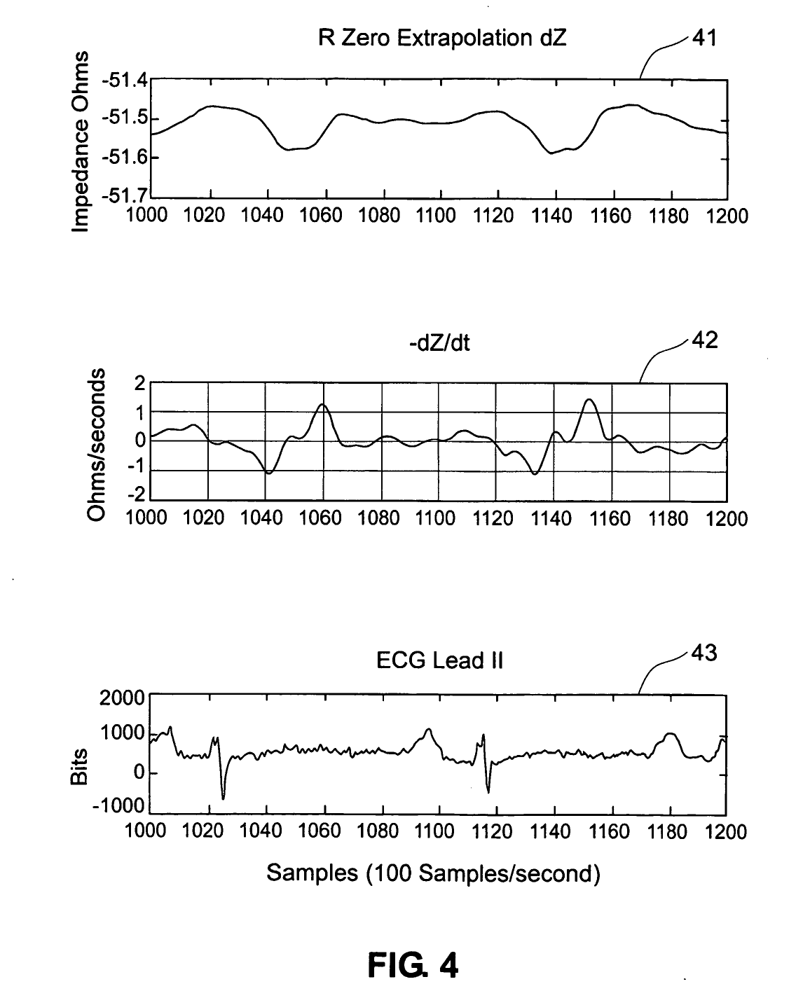

The final step (step 9) is to determine stroke volume and/or other measures of cardiac function. This can be done using the calculations of equation 3 or equation 4. The acquired data is conveniently displayed in the manner exemplified in FIG. 4.

The impedance is plotted 41 in FIG. 4 as a function of samples. The sampling rate for FIG. 4 is 100 samples per second so the x-axis is equivalent to 2 seconds of data.

To provide a time correlation an ECG 43 is recorded and displayed. It is clear that the traces in FIG. 4 cover approximately two cardiac cycles. The middle trace 42 is the time derivative dZ/dt of the impedance trace 41. The dZ/dt data is used to determine stroke volume (SV) and other measures of cardiac function.

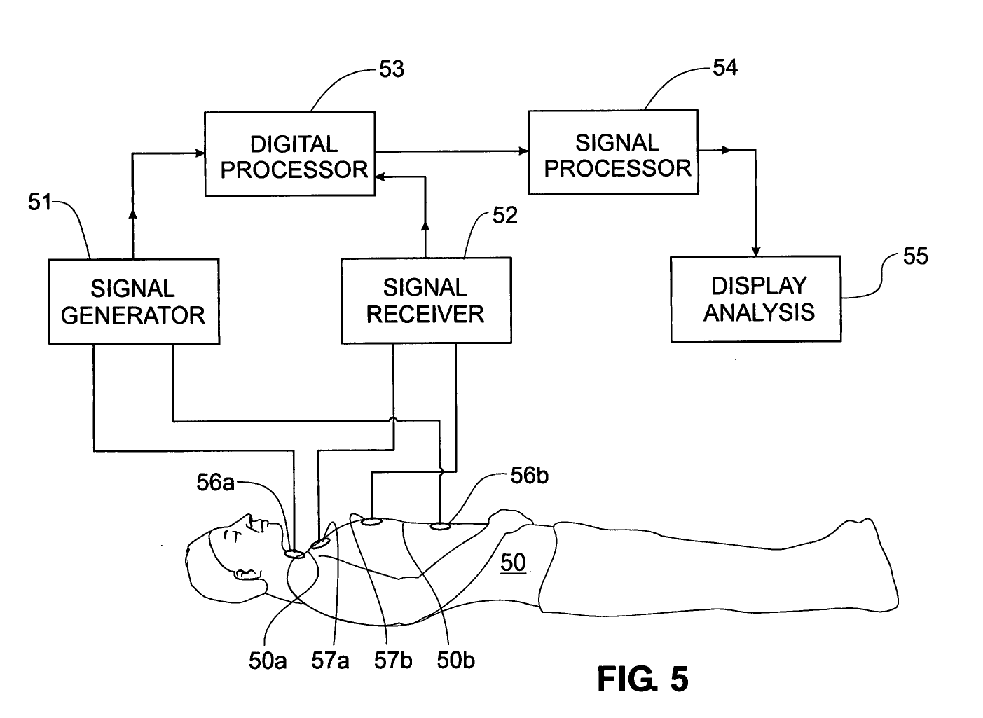

An apparatus suitable for working the method of FIG. 2 is shown schematically in FIG. 5. A signal generator 51 generates the constant current signal at multiple simultaneous frequencies referred to in step 1. The current is applied to a patient 50 using a pair of outer electrodes 56a and 56b attached to the thoracic region 50A and neck region 50B of patient 50.

A voltage is recorded by signal receiver 52 across a pair of inner electrodes 57a and 57b as referred to in step 2. A digital processor unit 53 performs data manipulation to present the current waveform and the voltage waveform in a suitable form to a signal processing unit 54. The signal processing unit performs steps 3 to 7 of the method of FIG. 2.

In one embodiment the signals generated by the signal generator 51 are fixed. The inventors have found that an embodiment is preferred in which the signal generator 51 is controllable to produce multiple selectable frequencies. That is, the number of different signals and the frequency of each signal are selectable. The selection is conveniently controlled by the digital processing unit 53.

The impedance data is displayed in the manner of FIG. 4 by display and analysis unit 55. The analysis includes steps 8 and 9 of FIG. 2. The data may also be stored for further later analysis.

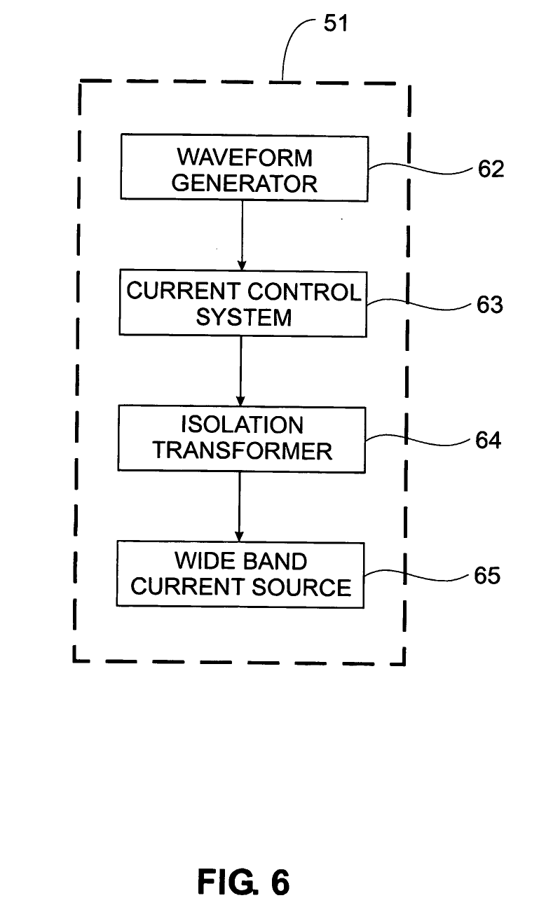

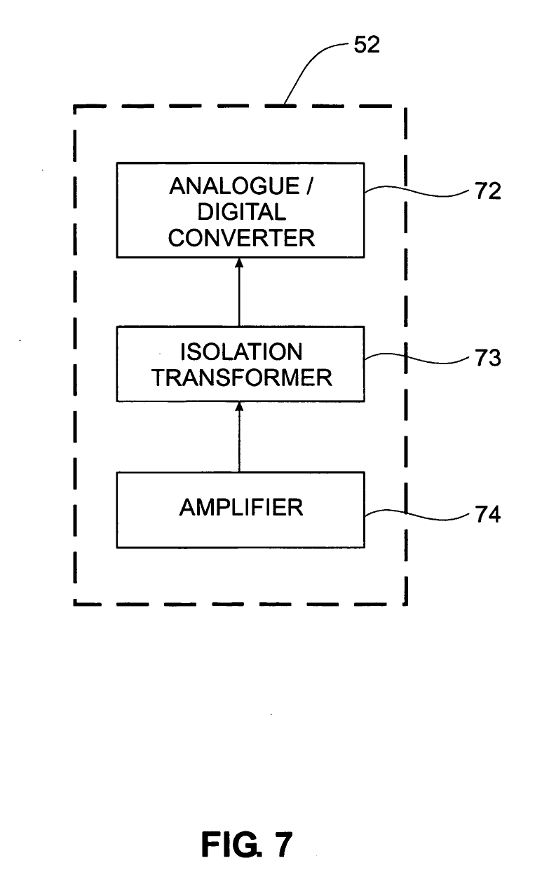

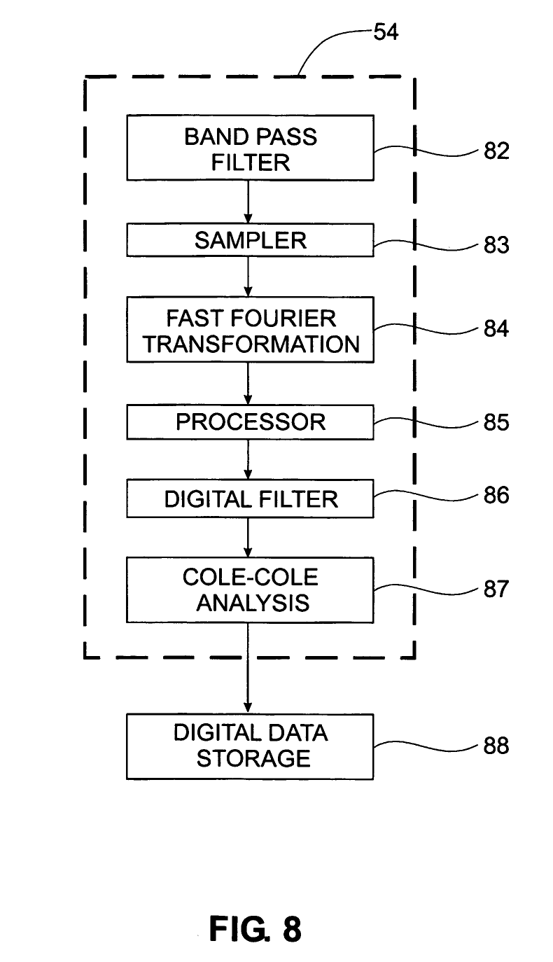

Further details of the signal generator 51 (step 1) are shown schematically in FIG. 6. A waveform generator 62 generates sinusoid signals at a range of selected frequencies (2-2000 kHz). The signals are applied to a wide band width current source 65 to produce the alternating current signal that is supplied to the electrodes.

Current control system 63 controls the current from waveform generator 62 and maintains constant current. An isolation transformer 64 protects patient 50 from any electrical fault in signal generator 51.

Outer electrodes 56a and 56b comprise circuitry for efficiently applying the current at various frequencies to patient 50. To facilitate attachment of electrodes 56a and 56b to patient 50, clips may be provided (not shown). Electrodes 56a and 56b also comprise shields to isolate any stray current from patient 50. The cables have a bandwidth sufficient to carry the range of frequencies at low current levels and have driven shields to minimize capacitive leakage.

Inner electrodes 57a and 57b measure the potential difference produced by the applied current from electrodes 56a and 56b through the tissue of thoracic region 50b of patient 50. Preferably, inner electrodes 57a and 57b are placed on opposite sides of the heart.

Inner electrodes 57a and 57b are connected to high input impedance amplifier 74 of signal receiver 52 (step 2) to amplify the recorded voltage. The signal output from amplifier 74 is fed into analogue to digital converter 72 through isolation transformer 73. Preferably, analogue to digital (A/D) converter 72 is a high bit, high speed AD converter, such as a 14 bit, 4 channel, 2.5 MS/s per channel A/D converter. The digitised signals are recorded (step 3) and then enter signal processing unit 54. Signal processing unit 54 also receives input from signal generator 51.

Before the impedance signals are demodulated (step 4) they are passed through band pass filter 82 and sampler 83. The signals are then converted to impedance frequency domains by Fast Fourier Transform (FFT) 84. FFT processor 84 performs FFT analysis on short time blocks of sampled bio-impedance data and individual frequencies are isolated to determine the impedance for each frequency, for each time block. The signal is converted into a two-dimensional function of time variable and a frequency variable.

Processing unit 85 receives the FFT frequency signals and performs an algorithm incorporating calibration coefficients to calibrate the measured impedances. A calibration card of circuits of known impedances can be provided which is used to calibrate the source and potential electrodes of the device. Signals produced by processing unit 85 are digitally-filtered by digital filter 86.

Electrocardiogram (ECG) electrodes (not shown) may also be attached to thoracic region 50b of patient 50 to obtain cardiographic signals of heart activity. The ECG signals are also fed into signal processing unit 54. The ECG is used to determine the electrical timing of the cardiac cycle to augment the information provided by the impedance signal. The ECG signal cuts data analysis time by identifying the data time blocks recorded before and during ventricular blood ejection. Preferably, the time period over which the FFT analysis is conducted begins just before the R wave peak of the heartbeat (ventricular contraction).

Digitally-filtered signals are plotted on a Cole-Cole plot (87) as described in step 6. The impedance data over the range of frequencies is made to fit the known theoretical circular locus. An impedance value at zero frequency Z₀ and also at infinite frequency Z_{inf} is extrapolated from the impedance spectrum. FIG. 3 is an example of a Cole-Cole plot.

Z₀ is the theoretical impedance to a DC signal as shown in FIG. 3 and corresponds to the impedance of extracellular fluid or water (ECW). The ECW impedance values can be plotted with respect to time and correlated to the ECG signal.

Cole-Cole analysis 87 can also derive the change of impedance Z over time, the rate of change of the measured impedance at the systolic cycle of the heart, dZ/dt to determine impedance parameters Z₀ (baseline impedance), dZ/dtₘₐₓ and LVET.

The cardiac output (stroke volume multiplied by heart rate) is obtained by calculating either equation 3 or 4 using the parameters obtained above at steps 6 and 7. The equations provide the stroke volume values of the heart. However, the above parameters can be further processed to determine other cardiac output parameters indicative of heart activity, such as ejection fraction. All digital data can be stored on data storage unit 88.

The inventors have found that the Cole-Cole analysis methodology is useful but the invention is not limited to this technique. Other techniques such as Bode analysis or Argand analysis are also suitable.

The present invention provides an improved bio-impedance device which measures cardiac output using multiple frequencies to determine impedances, and to calculate the changes in extracellular fluid volume (blood volume) in each time block.

The invention has been described with reference to an exemplary embodiment. However, it should be noted that other embodiments are envisaged within the scope and spirit of the invention.

The advantages of the impedance cardiography device of this invention are as follows:
(i) it is a non-invasive technique and therefore exposes the patient to fewer risks and less pain and stress;
(ii) the system is portable and can therefore be easily transported to isolated and rural areas;
(iii) the device does not require expert operator skills;
(iv) use of the device with an electrocardiogram results in a complete cardiac assessment of the patient and results are obtained immediately;
(v) it can be performed on virtually all subject groups including the critically ill, elderly, infants and pregnant individuals;
(vi) it can be performed in both clinical hospital settings as well as GP surgeries; and
(vii) the use and implementation of this device will reduce national health care costs dramatically.

1. A method of determining measures of cardiac function in a patient including the steps of;
(i) generating an alternating current signal at multiple simultaneous frequencies from a constant current source electrically isolated from the patient;
(ii) applying the current to an outer pair of electrodes on the patient;
(iii) measuring a voltage signal across an inner pair of electrodes on the patient;
(iv) demodulating the current signal and voltage signal to extract signals at each of said multiple frequencies;
(v) determining impedance at each said frequency at a time;
(vi) fitting said impedance at each frequency to a theoretical frequency dependent impedance locus;
(vii) extrapolating the locus to obtain a value of impedance at zero frequency at said time;
(viii) repeating steps (v) to (vii) to obtain a time varying plot of impedance; and
(ix) calculating measures of cardiac function in the patient from said time varying plot.

2. The method of claim 1 wherein said multiple simultaneous frequencies comprise at least three frequencies of stimulation.

3. The method of claim 1 wherein said multiple simultaneous frequencies comprise at least five frequencies of stimulation.

4. The method of claim 1 wherein said frequencies fall within the range 2-2000 kHz.

5. The method of claim 1 wherein said frequencies fall within the range 10-500 kHz.

6. The method of claim 1 wherein the frequency and waveform of the alternating current signal is selectable or fixed.

7. The method of claim 1 wherein the current signal and the voltage signal are demodulated using Fast Fourier Transform.

8. The method of claim 1 wherein the Fast Fourier Transform of said current signal and said voltage signal provides a phase value and an amplitude value from which impedance is determined.

9. The method of claim 1 further including the step of recording an ECG and correlating the ECG with the time varying plot of impedance.

10. The method of claim 1 wherein the change in the impedance value over time and the rate of change in the measured impedance signal dZ/dt is used to determine impedance parameters to calculate cardiac output of said patient.

11. The method of claim 1 wherein a time derivative of said impedance signal is mathematically obtained using the extrapolated impedance at zero frequency (Z₀) or at infinite frequency (Z_{inf}).

12. The method of claim 1 wherein the theoretical frequency dependant impedance locus is a Cole-Cole analysis.

13. The method of claim 1 wherein steps (i) to (viii) are repeated to record at least one cardiac cycle.

14. The method of claim 1 wherein measures of cardiac function are calculated using the following equation:
$$SV = \frac{ρ {L}^{2} {〈 \frac{ⅆ Z}{ⅆ t} 〉}_{max} VET}{{Z}_{B}^{2}}$$
where: SV=stroke volume
(dz/dt)ₘₐₓ=maximum rate of change in measured impedance at the beginning of systolic cycle
VET=left ventricular ejection time.

15. The method of claim 1 wherein measures of cardiac function are calculated using the following equation:
$$SV = \frac{ {L}^{t 3} {〈 \frac{ⅆ Z}{ⅆ t} 〉}_{max} VET}{{Z}_{B}}$$
where: SV=stroke volume
(dz/dt)ₘₐₓ=maximum rate of change in measured impedance at the beginning of systolic cycle
VET=left ventricular ejection time
L′=thoracic length estimated from the subject's height and weight using a nomogram
L′=blood resistivity.

16. The method of claim 1 further including the step of measuring and recording the distance between the inner electrodes.

17. The method of claim 1 further including the step of measuring and recording the height, weight, sex and age of the patient.

18. The method of claim 1 wherein the steps of demodulating and determining an impedance at a time, comprises the steps of:
sampling the impedance signals to obtain a sampled impedance;
applying a time to frequency domain transform to said sampled signal to obtain transformed impedance signals; and
filtering the transformed impedance signals and isolating each frequency to determine the impedance for each frequency at each time.

19. An apparatus for non-invasive measurement of cardiac function in a patient, said apparatus comprising:
a constant current source, electrically isolated from said patient, generating an alternating current signal at multiple simultaneous frequencies, which is applied to an outer pair of electrodes on a patient;
an inner pair of electrodes applied to a patient for measuring a voltage signal;
signal processing means for converting said applied current signal and measured voltage signal to impedance signals at each frequency at a time;
means for determining impedance values at a zero frequency (Z₀) and at infinite frequency (Z_{inf}) at a plurality of time intervals; and
means for calculating measures of cardiac function in said patient from said impedance values.

20. The apparatus of claim 19 wherein said outer pair of electrodes comprise shields to protect the patient from stray current.
